(19) United States
(12) Patent Application Publication
(10) Pub. No.: US 2024/0415670 A1
(43) Pub. Date: Dec. 19, 2024

(54) PLIF SPACER

(71) Applicant: ATLAS SPINE, INC., Jupiter, FL (US)

(72) Inventor: Matthew G. Baynham, Jupiter, FL (US)

(21) Appl. No.: 18/742,374

(22) Filed: Jun. 13, 2024

Related U.S. Application Data
(60) Provisional application No. 63/507,885, filed on Jun. 13, 2023.

(51) Int. Cl. A61F 2/44; A61F 2/30

(52) U.S. Cl. CPC A61F 2/446; A61F 2002/3011

(57) ABSTRACT

An implant having an upper support body and a lower support body that are coupled together by use of pinions integral to the upper support body and apertures integral to the lower support body. A dual directional driver is rotatably coupled to a forward spacer and a rear spacer, wherein the implant forms a compact arrangement for ease of insertion allowing a posterior surgical approach for placement of the spinal implant for interbody fusion allowing the implant to be inserted through a small incision and rotation of the dual directional driver will cause movement of the upper support body an equal distance from the lower support body to increase the implant in size in situ.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 63/507,885 entitled “PLIF SPACER”, filed Jun. 13, 2023; the contents of which is incorporated herein by reference in its entirety.

### FIELD OF THE INVENTION

This invention relates to the field of orthopedic surgery and, more particularly, to implants to be placed between vertebrae in the spine.

### BACKGROUND OF THE INVENTION

Spinal stabilization is one approach to alleviating chronic back pain caused by displaced disk material or excessive movement of individual vertebrae. Conventional stabilization techniques include fusing two or more vertebrae together to circumvent or immobilize the area of excessive movement. Normally, the vertebral disk material which separates the vertebrae is removed and bone graft material is inserted in the space for interbody fusion. In addition to or in place of the bone graft material, a spinal implant may be inserted in the intervertebral space. Posterior Lumbar Interbody Fusion (PLIF), which is a surgical procedure that involves removing a portion of the damaged disc in the lower back and replacing it with a spacer to restore the height and stability of the spinal column. PLIF spacers are used to fill the empty space left after the removal of the damaged disc.

During the PLIF procedure, a surgeon makes an incision in the lower back and moves the muscles and other tissues aside to access the affected disc. The damaged portion of the disc is then removed, and the PLIF spacer is inserted into the empty space. The spacer helps to restore the proper alignment and spacing of the vertebrae, which can relieve pressure on the nerves and reduce pain. Over time, the patient's body will naturally grow new bone tissue around the PLIF spacer, fusing the vertebrae together into a single solid bone. This fusion helps to stabilize the spinal column and reduce the risk of future injury or degeneration. Over the years, PLIF spacers have been refined and improved to enhance their effectiveness and safety. Today, they are widely used in the treatment of a variety of spinal conditions, including degenerative disc discs, spinal stenosis, and disease, herniated spondylolisthesis.

Applicant has numerous issued patent registrations pertaining to PLIF devices including: U.S. Pat. Nos. 11,491,024; 10,226,360; 9,889,020; and 9,421,111; the contents of which are incorporated herein by reference.

### SUMMARY OF THE INVENTION

An implant having an upper support body and a lower support body that are coupled hinged together by use of pinions 16 and 18 integral to the upper support body and apertures integral to the lower support body. A dual directional driver is rotatably coupled to a forward spacer and a rear spacer, wherein the implant forms a compact arrangement for ease of insertion allowing a posterior surgical approach for placement of the spinal implant for interbody fusion allowing the implant to be inserted through a small incision and rotation of the dual directional driver will cause movement of the upper support body an equal distance from the lower support body to increase the implant in size in situ. The upper support body is formed from a substantially flat plate having an upper surface to provide a large contact area with the vertebra. The upper surface with contact lands and grooves to provide better purchase. The upper support body is further defined by a first front side inclined surface spaced from a first rear side. A second front side inclined surface forms a mirror image of the first front side inclined surface. The lower support body is formed from a substantially flat plate having an outer surface to provide a large contact area with the vertebra. The lower upper support body is further defined by a first lower front side inclined surface spaced from a first lower rear side inclined surface. The first lower front side inclined surface is spaced apart from a first lower rear side inclined surface by a pinion receptacle. A second lower front side inclined surface forms a mirror image of the first lower front side inclined surface. Similarity a second lower rear side inclined surface forms a mirror image of the first lower rear side inclined surface. A cylindrical shank driver having left hand threads is coupled to the forward spacer having and a rear spacer. Rotation of the driver pulls the spacers across the inclined surfaces formed in the upper and lower support body providing an even spacing of the flat plates.

An objective of the invention is to provide a spinal implant with a simplistic design that has a low manufacturing cost and robust elements.

Another objective of the invention is to provide a PLIF implant using a threaded driver with rotating threads to assure even spacing of an upper and lower support body.

Other objectives and advantages of this invention will become apparent from the following description taken in conjunction with any accompanying drawings; wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is an exploded view of the spinal implant;

FIG. 2 is a perspective view of the spinal implant in a compressed position;

FIG. 3 is a top view thereof;

FIG. 4 is a side view thereof;

FIG. 5 is a rear end view thereof;

FIG. 6 is a front view thereof; and

FIG. 7 is a perspective of the spinal implant in an expanded position.

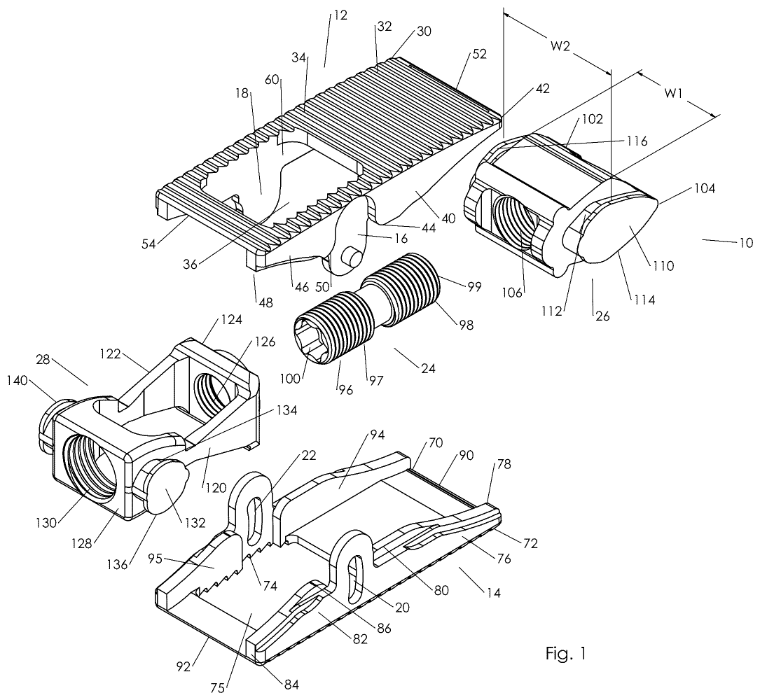

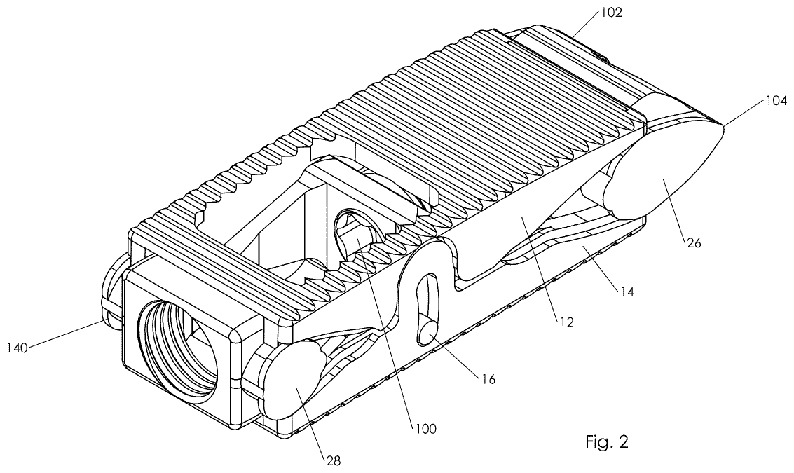

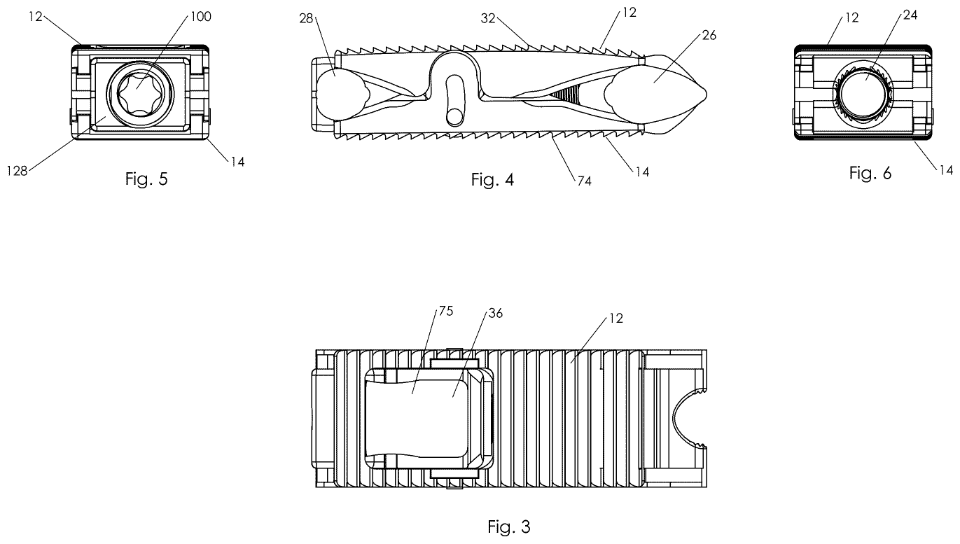

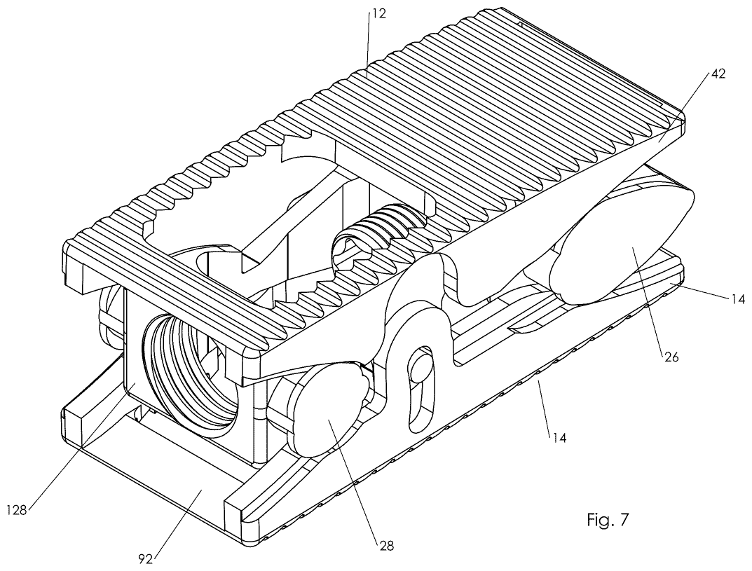

### DETAILED DESCRIPTION OF THE INVENTION

The spinal implant 10 is inserted in the intervertebral space to replace damaged, missing or excised disk material. This extended position allows the leading end of the distractor to be inserted in a small intervertebral space without the necessity of excising structurally sound bone.

Referring to the Figures, disclosed is an implant 10 defined by an upper support body 12 and a lower support body 14 that are coupled together by use of pinions 16 and 18 integral to the upper support body insertable to apertures 20 and 22 integral to the lower support body. A dual directional driver 24 is rotatably coupled to a forward spacer 26 and a rear spacer 28, wherein the implant forms a compact arrangement for ease of insertion allowing a posterior surgical approach for placement of the spinal implant for interbody fusion allowing the implant to be inserted through a small incision and rotation of the dual directional driver 24 will cause movement of the upper support body 12 an equal distance from the lower support body 14 to increase the implant 10 in size in situ.

The upper support body 12 is formed from a substantially flat having an upper surface 32 to provide a large contact area with the vertebra. The upper surface 32 with contact lands and grooves 34 to provide better purchase, although other stippled treatment may be employed. An aperture 36 provides spacing for new bone tissue to secure the plate 30. The upper support body 12 is further defined by an a first front side inclined surface 40 having a front edge 42 sloped toward a center edge 44 and spaced from a first rear side inclined surface 46 having a rear edge 48 sloped to a quarter edge 50, the front side inclined surface 40 spaced apart from a first rear side inclined surface 46 by the pinion 16, the plate 30 forming a proximal end 52 and a distal end 54. A second front side inclined surface 60 forms a mirror image of the first front side inclined surface 40.

The lower support body 14 is formed from a substantially flat plate 70 having an outer surface 72 to provide a large contact area with the vertebra. The outer surface 72 with contact lands and grooves 74 to provide better purchase. An aperture 75 provides spacing for new bone tissue to secure the plate 70. The lower upper support body 14 is further defined by an a first lower front side inclined surface 76 having a front edge 78 sloped toward a center edge 80 and spaced from a first lower rear side inclined surface 82 having a rear edge 84 sloped to a quarter edge 86, the first lower front side inclined surface 76 spaced apart from a first lower rear side inclined surface 82 by the pinion receptacle 20, the plate 70 forming a proximal end 90 and a distal end 92. A second lower front side inclined surface 94 forms a mirror image of the first lower front side inclined surface 76. Similarity a second lower rear side inclined surface 95 forms a mirror image of the first lower rear side inclined surface 82. The outer surface 72 with contact lands and grooves 74 to provide better purchase, although other stippled treatment may be employed.

In a preferred embodiment, the above mentioned inclined surfaces include a radius wherein the forward and rearward spacer operates in combination with the respective inclined surface providing a cam effect. For instance, the dual directional driver 24 may cause the forward and rearward spacer to be drawn at the same rate and the radius of the inclined surface can be constructed and arranged to cause more lift in the front of the device and less in the rear of the device. The inclined surfaces and spacers provide a mechanical advantage such as rapid expansion in the beginning of spacer movement and more power towards the end of the spacer movement.

The spacer may be constructed in the shape of a cam surface, the cam surface designed to impart a change in the operational characteristics of the spacer wherein the cam is calculated in combination with the inclined surface to provide lift with mechanical advantages at various positions of the dual directional driver.

In another embodiment, the threads on the dual directional driver may be constructed to impart front spacer movement to be at a different rate than the rear spacer. For example, the threads per inch on the front threads of the dual directional driver may be less than the threads per inch on the rear threads of the directional driver. In this example, the change in threads per inch would cause the front spacing to rise at a faster rate than the rearward spacing. Similarly, the directional driver may have a ratio pitch wherein the rearward spacing could rise at a faster rate than the forward spacing.

The driver 24 is a cylindrical shank 94 having left hand threads 96 on a first end 97 and right hand threads 98 on a second end 99 with a drive socket 100 formed in the first end 97. The driver 24 is coupled to the forward spacer 26 having a body 102 with a width W1 positionable with the side walls. Right inclined surface protrusion 110 having a upper surface 112 for engaging the first front side inclined surface 40 and a lower surface 114 for engaging the first lower front side inclined surface 76. Left inclined surface protrusion 116 forming a mirror image of the right inclined surface protrusion 110.

The driver 24 is further coupled to the rear spacer 28 defined by side wall 120, 122 spaced apart by a leading wall 124 having a threaded opening 126 for coupling to the first end 97 of the driver 24. Side walls 120, 122 further spaced apart by trailing wall 128 having threaded receptacle 130 for receipt of a positioning tool. Right rear spacer protrusion 132 having an upper surface 134 and a lower surface 136 for engaging the first upper rear side inclined surface 46 and the first lower rear side inclined surface 82. Left rear spacer protrusion 140 forming a mirror image of the right rear spacer protrusion 132.

A positioning tool, not shown, is temporarily secured to the rear spacer 28 for ease of placement. The implant 10 is constructed and arranged to slide between vertebra after a discectomy. Once positioned a driver tool, not shown, engages the driver 24 and upon rotation of the driver tool, the front spacer 26 and the rear spacer 28 are drawn together causing the upper plate 12 and lower plate 14 to expand for restore proper spinal alignment and height. The apertures 36, 75 are open to enhance fusion with additional space between the plates to receive gone grafts or substitutes to promote fusion of the implant. The implant 10 is preferably constructed from titanium, polyether ether ketone (PEEK), or a carbon-fiber reinforced polymer.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A spinal implant comprising:
an upper support body formed from a substantially flat plate having an upper surface and a lower surface, said lower surface having a first front side inclined surface spaced from a first rear side inclined surface spaced apart from a second front side inclined surface by a pinion support, a second front side inclined surface is spaced apart from a second rear side inclined surface forming a mirror image of said first front side inclined surface and said first rear side inclined surface;
a lower support body formed from a substantially flat plate having an outer surface, said lower support body having a first lower front side inclined surface spaced from a first lower rear side inclined surface by a pinion receptacle; a second lower front side inclined surface forms a mirror image of the first lower front side inclined surface and said first lower rear side inclined surface;
a driver formed from a cylindrical shank having left hand threads along a first end and right hand threads along a second end, said first end including a driver tool receptacle;
a forward spacer coupled to said driver, said forward spacer positionable between said upper and lower front inclined surfaces having inclined surface protrusion for engaging the front side inclined surfaces;
a rear spacer having a threaded opening for coupling to the first end of the driver with side walls spaced apart by trailing wall having a threaded receptacle for receipt of a positioning tool, a right rear spacer protrusion having an upper surface and a lower surface for engaging the first upper rear side inclined surface and the first lower rear side inclined surface, with a left rear spacer protrusion forming a mirror image of the right rear spacer protrusion;
wherein the implant forms a compact arrangement for ease of insertion allowing a posterior surgical approach for placement of said spinal implant for interbody fusion, said driver tool receptacle for use in rotation of said driver allowing movement of the upper support body an equal distance from the lower support body to increase the implant in size in situ.

2. The spinal implant according to claim 1 wherein said driver right and left hand threads are reversed.

3. The spinal implant according to claim 1 wherein each said inclined surface includes a radius.

4. The spinal implant according to claim 1 wherein each said spacer is cam shaped.

5. The spinal implant according to claim 1 wherein said driver right hand threads has a first amount of threads per inch and said driver left hand threads has a second amount of threads per inch.

6. The spinal implant according to claim 1 wherein an upper surface of said upper support body and an outer surface of said lower support body are stippled.

7. The spinal implant according to claim 1 wherein said insert body is constructed from polyether ether ketone.
